(19) United States
(12) Patent Application Publication
(10) Pub. No.: US 2018/0125480 A1
(43) Pub. Date: May 10, 2018

(54) Suture-Passing Laparoscopic Knot Tying Instrument

(71) Applicant: Peter Fan, Englewood Cliffs, NJ (US)

(72) Inventor: Peter Fan, Englewood Cliffs, NJ (US)

(21) Appl. No.: 15/859,717

(22) Filed: Jan. 1, 2018

(51) Int. Cl. A61B 17/04

(52) U.S. Cl. CPC A61B 17/0485; A61B 2017/0474; A61B 17/0469

(57) ABSTRACT

This instrument is intended to facilitate intra-corporeal laparoscopic tying of knots. The shafts of two small diameter laparoscopic graspers are combined within a common external sheath, and both are joined to a common handle, to be used by one hand. One grasper has no handle, but is able to rotate, and acts as a donor grasper, whilst the other has a regular scissors type of handle, is stationary, and acts as a recipient grasper. The instrument enables the head end of a suture to be passed from a donor grasper to an adjacent recipient grasper, passing behind and around its tail strand, forming the knot, in the same manner as the tying of shoe-laces.

### FIELD OF THE INVENTION

The tying of knots in a suture intra-corporeally with laparoscopic instruments is difficult and time consuming. Surgeons still need an instrument that would facilitate this tying process. The present device enables passing of the head end of a suture from a donor grasper to an adjacent recipient grasper behind the tail end of the same suture, thereby creating the knot.

### BACKGROUND OF THE INVENTION

Tying of knots is essential in any kind of surgery. It is relatively easy in open surgery, but is difficult in laparoscopic surgery. The current art of laparoscopic knot tying employs either the extra-corporeal method, involving tying the knot by hand outside the body and pushing the knot inside with a knot pusher, or the intra-corporeal method, involving manipulation of the suture with the tips of two laparoscopic graspers, which is slow and cumbersome, and requires considerable skill. Laparoscopic clip appliers, staplers, pre-tied knots and the like are useful substitutes, but cannot totally replace tied knots which are still needed. Despite considerable prior art, today hardly any hand operated instrument exists that renders laparoscopic intra-corporeal knot tying easier and faster.

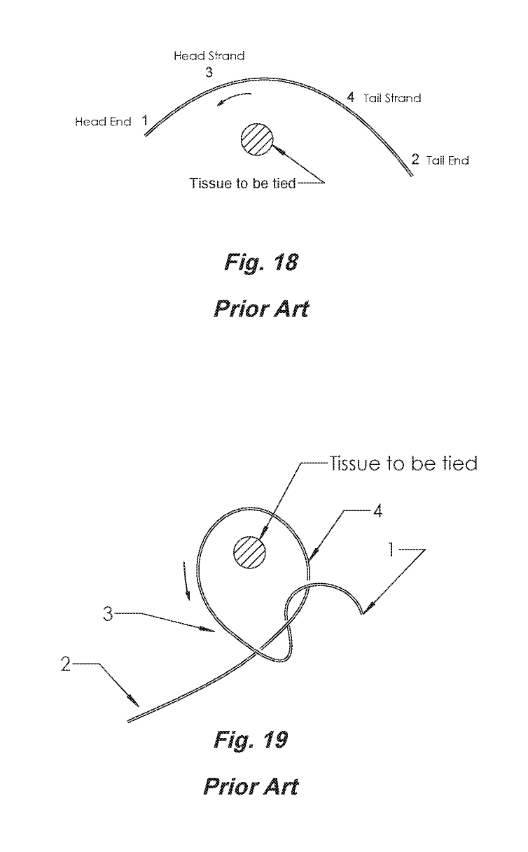

In order to describe the tying process, the different parts of a suture ligature need first be given names. As shown in FIG. 18 in the drawings, after the suture ligature has passed around the tissue to be tied, it then presents with a head end (1), a tail end (2), a leading strand (3), and a tail strand (4).

There are three basic methods of tying a knot, whether done openly or laparoscopically. The first method makes the head end of the suture pass 360 degrees continuously around the tail strand, as is shown in the U.S. Pat. No. 9,561,028, “Automatic Laparoscopic Knot Tier”, invented by this author, and was designed specifically to avoid the release and re-grab.

The second method makes a loop, which is commonly used by surgeons performing open surgery, where the surgeon makes “instrument ties”, by wrapping the tail strand of the suture around the needle holder, and then pulling the head end of the suture through the loop. The instrument described in the recent U.S. Pat. No. 9,820,736, invented also by this author, makes such a loop laparoscopically.

The third method involves making a “throw”, which passes the head end of the suture behind its tail strand, between two adjacent graspers. This simulates the tying of shoe-laces by fingers, and requires the release and re-grab of the head end of the suture, behind the tail strand, which is now the object of the present invention.

In the present invention, two small diameter laparoscopic graspers are incorporated inside a common external sheath, with one being stationary and keeping a regular scissors type of handle, known as the recipient grasper, and the other losing its handle, becoming rotatable, known as the donor grasper.

Referring to the author's own previous attempts, the first was the “Double Laparoscopic Grasper”, U.S. Ser. No. 13/051,992, which was abandoned because the passing of the suture between the two graspers could not be accomplished at that time. The author's second attempt was the “Automatic Laparoscopic Knot Tying Instrument”, U.S. Pat. No. 9,561,028, which uses a mini-grasper at the tip of the instrument grasping the head end of the suture, then rotating through 360 degrees around the tail strand, and avoiding the release and re-grab of the head end of the suture. The author's third attempt was the recent “Laparoscopic Suture Loop Maker”, U.S. Pat. No. 9,820,736, which worked quite well, but also avoided the release and re-grab. However, the 2^{nd} and 3^{rd} inventions were not fully practicable because, after the knot was made, the final take away depended on springs holding on to the head end of the suture, which proved inadequate. The current invention improves the holding power of the jaws by using thumb pressure with a regular scissors type of handle.

Referring to the previous literature, the Christoudias Double Grasper has 3 jaws, with a common middle jaw, but functions as a tissue approximator. Its spring-loaded actuators are operated by two push buttons. The Ferzli Double Grasper, has a second pair of jaws positioned more proximally on the main shaft, whose purpose is to anchor one end of the suture prior to twisting it around the shaft of the instrument in order to produce a loop. The Hasson Suture Tying Forceps, is similar to the Ferzli, with 3 finger loops. The orthopedic suture passers are for passing sutures only through hard tissue, and these include the Arthrex Scorpion Suture Passer, and the Arthrex Birdbeak Suture Passer. Some suture passers are for passing sutures through a thickness of soft tissue such as the abdominal wall, and these include the Goretex and the Aesculap. There are devices which “pass the suture-needle” side to side, for inserting sutures into tissues, as well as for tying knots, e.g. the Autosuture's Endo-stitch, and the Japanese Maniceps. Note these only pass the suture needle, not the suture thread per se. There have been devices that attempt to “automatically” tie a knot, such as Jerrigan's experimental rotating slotted disc designed for robotic endo-cardiac surgery, but it was abandoned because of the requirement for a manufactured cartridge.

There have been also many devices that help to “create a loop”, but with each functioning differently—(a) Kitano's grasper with the rotating sleeve, Japanese, (b) Donald Murphy's grasper with the extra horn, Australian, (c) Grice's sleeve catching instrument, (d) Bagnato & Wilson's device which simulates the radiological pig-tail catheter, with a preformed loop built into the tip of the catheter, which is deformable and purportedly a loop former, but it is difficult to manufacture and apply, and has not yet been reduced to practice, (e) Ferzli's double grasper, which anchors one end of the suture, as described above. There have been devices using a “pre-formed knot”, (1) Ethicon's Endo-Loop, (2) the Duraknot, (3) LSI's device, (4) Pare's pre-tied knot, all of which do not help to tie knots.

Other past inventions related to intra-corporeal laparoscopic knot tying fail to address the basic problem of “how to create a knot”. They usually offer various alternatives, such as making fishing knots, using pre-tied knots, knot pushers, suture clips, cinchers, tissue fasteners, anchors, stapling devices, etc. The present invention however passes the head end of the suture behind its tail end, to make the actual knot intra-corporeally.

### U.S. PATENT DOCUMENTS

| | | | | |
|---|---|---|---|---|
| 1. | 3834395 | Sep. 10, 1974 | Manuel Santos | 128/326 |
| 2. | 5201759 | Apr. 13, 1993 | George Ferzli. | 606/139 |
| 3. | 5217471 | Jun. 8, 1993 | Stephen Burkhart | 606/148 |
| 4. | 5234443 | Aug. 10, 1993 | Phan & Stoller | 606/148 |
| 5. | 5250054 | Oct. 5, 1993 | Lehmann Li | 606/148 |
| 6. | 5281236 | Jan. 25, 1994 | Bagnato et al. | 606/139 |
| 7. | 5312423 | May 17, 1994 | Rosenbluth & Brenneman | 606/148 |
| 8. | 5395382 | Mar. 7, 1995 | DiGiovanni et al. | 606/148 |
| 9. | 5437682 | Aug. 1, 1995 | Drew Grice | 606/148 |
| 10. | 5423836 | Jun. 13, 1995 | Scott Brown | 606/148 |
| 11. | 5439467 | Aug. 8, 1995 | Theodore Benderev, et al. | 606/139 |
| 12. | 5480406 | Jan. 2, 1996 | Nolan et al. | 606/139 |
| 13. | 5728109 | Mar. 17, 1998 | Schulze et al. | 606/148 |
| 14. | 5810852 | Sep. 22, 1998 | Greenberg et al. | 606/148 |
| 15. | 5814054 | Sep. 29, 1998 | Kortenbach et al. | 606/139 |
| 16. | 5846254 | Dec. 8, 1998 | Schulze et al. | 606/228 |
| 17. | 6051006 | Apr. 18, 2000 | Shluzas & Sikora | 606/148 |
| 18. | 6086601 | Jul. 1, 2000 | InBae Yoon | 606/148 |
| 19. | 6221084 | Apr. 24, 2001 | R. Fleenor, Pare Surgical | 606/148 |
| 20. | 6432118 | Aug. 13, 2002 | Mollenhauer & Kucklick | 606/148 |
| 21. | 6716224 | Apr. 26, 2004 | Singhatat | 606/148 |
| 22. | 2009/0228025 | Sep. 10, 2009 | Steven Benson | 606/144 |
| 23. | 2010/0016883 | Jan. 21, 2010 | George Christoudias | 606/205 |
| 23. | 5312423 | May 17, 1994 | Rosenbluth et al. | 606/148 |
| 25 | 8512362 | Aug. 20, 2013 | Ewers et al. | 606/158 |
| 26. | 4635638 | January 1987 | Weintraub. | |
| 27. | 5938668 | August 1999 | Scirica. | |
| 28. | 5954731 | September 1999 | Yoon. | |
| 29. | 6017358 | January 2000 | Yoon | |
| 30. | 6086601 | July 2000 | Yoon. | |
| 31. | 2008/0228204 | September 2008 | Hamilton. | |

| | | | |
|---|---|---|---|
| 32. | 2013/051992 | Sep. 30, 2012 | Fan - Double Laparoscopic Grasper. |
| 33. | 9561028 | Feb. 7, 2017 | Fan - Automatic Lap. Knot |
| | | | Instrument |
| 34. | 9820736 | Nov. 21, 2017 | Fan - Laparoscopic Suture Loop |
| | | | Maker. |

### OTHER PUBLICATIONS

1. Endo-stitch—Autosuture—Manufacturer's item #173016.

2. Maniceps—Japanese suturing device, similar to Endo-stitch.

3. A Laparoscopic Device for Minimally Invasive Cardiac Surg. Shaphan Jernigan, et. al.—European J. of Cardio-thoracic Surgery, Vol. 37, p. 626-630. March 2010.

4. Knot Tying Intra-corporeally, with newly designed Forceps. (sliding sleeve).

5. Kitano et. al.—J. of Minimal Invasive Therapy & Allied Tech, 1996. 5: 27-28.

6. Endoscopic Knot Tying Made Easier—(one jaw with extra bump).

7. Donald Murphy—ANZ J. Surg. 1995. 65, 507-509.

8. The Excalibur Suturing Needle Holder—(jaw with prominent heel, helps looping)

9. Uchida et. al. Surgical Endoscopy—vol. 3, 531-532

10. Alijizawi laparoscopic auto-knot device—(two dissolving balls).

11. A New Reusable Instrument designed for simple and secure knot tying in laparoscopic surgery. S. S. Miller 1996 Surg. Endos 10: 940-941 (pointed canula).

12. The Nobel Automatic Laparoscopic Knotting and Suturing Device. Mishra et. al. World Laparoscopy Hospital, India. (a knot pusher)

13. Automated Knot Tying for Fixation in Minimally Invasive Robot Assisted Cardiac Surgery. March 9(1):105-12.

14. Kuniholm & Buckner—J. Biomed Eng. November 2005, Vol. 127, 1001-8. JSLS. 2005 Jan. 17.

15. M I Frecke—Laparoscopic multifunctional instruments: design and testing. Endosc Surg Allied Technol. 1994 December; 2(6):318-9.

16. G. Berci—Multifunctional laparoscopic Instruments.

17. http://www.ligasure.com/ligasure/pages.aspx?page=Products/Laparoscopi

18. http://www.freepatentsonline.com/y2010/0063437.

19. http//www.ncbi.nlm.nih.gov/pubmed/15791983 Multifunctional Laparoscopic Instruments.

| | |
|---|---|
| SUGGESTED U.S. | 606/139, 144, 145, 148. |
| CLASSIFICATION: | |
| SUGGESTED | A61B 17/00, 04, 28. |
| INTERNATIONAL | |
| CLASSIFICATION: | |
| FIELD OF SEARCH: | 606/139, 144, 145, 147, 148, 150, 151, 127,128, |
| | 606/167, 168, 170, 174, 182, 185, 205, 207, |
| | 210, 211. |
| RELATED PRIOR | 2013/051,992 9194468. 9561028. 9820736. |
| PATENTS: | |

### SUMMARY OF THE INVENTION

In laparoscopic surgery, the tying of knots intra-corporeally is still technically difficult and requires considerable skill and practice. The advent of laparoscopic clips and staples has been a great blessing to surgeons, but cannot totally replace the use of tied knots, which is still necessary. The instrument presented here enables the passing of the head end of suture between two adjacent graspers, behind and around the tail end of the suture, thus forming a knot. It conforms to the customary shape and size of a laparoscopic instrument, with an elongated round sheath, a regular scissors type of handle at the proximal end, and two small diameter grasper tips protruding at the distal end. The jaws of the graspers are controlled manually, and by compression springs. The main grasper, known as the recipient grasper, is stationary and non-mobile, and behaves like a regular grasper, while the secondary grasper, known as the donor grasper is without a handle, but is rotatable. The combination of the two of special mini-graspers with the special handle, enables the passing of the suture and is the heart of this device. All the manipulations at the handle are performed by the thumb, namely: opening the left jaw of the donor grasper, rotating the donor grasper, and closing the upper jaw of the recipient grasper.

### PART NUMBERS AND NAMES

| | | | |
|---|---|---|---|
| 1. | Stationary Handle. | 2. | Movable Handle. |
| 3. | Adaptor-2. | 4. | Rotation Lever. |
| 5. | Button for Lever. | 6. | Adaptor-1. |
| 7. | Ball. | 8. | Rod-Extension. |
| 9. | Button for Rod-Extension. | 10. | Retaining Flange Nut. |
| 11. | Front Spacer. | 12. | Rear Spacer. |
| 13. | Main Sheath. | 14. | Sheath-1. |
| 15. | Sheath-2. | 16. | Rod-1. |
| 17. | Rod-2. | 18. | Mini-Screw for Adaptors. |
| 19. | Compression Spring. | 20. | Mini-Screw for Lid. |
| 21. | Lid. | 22. | Link for jaws. |
| 23. | Movable upper jaw, | 24. | Fixed lower jaw. |
| 25. | Pin. | 26. | Male Hinge Bolt. |
| 27. | Female Hinge Bolt. | | |

### DETAILED DESCRIPTIONS OF THE DRAWINGS

Sheet 1

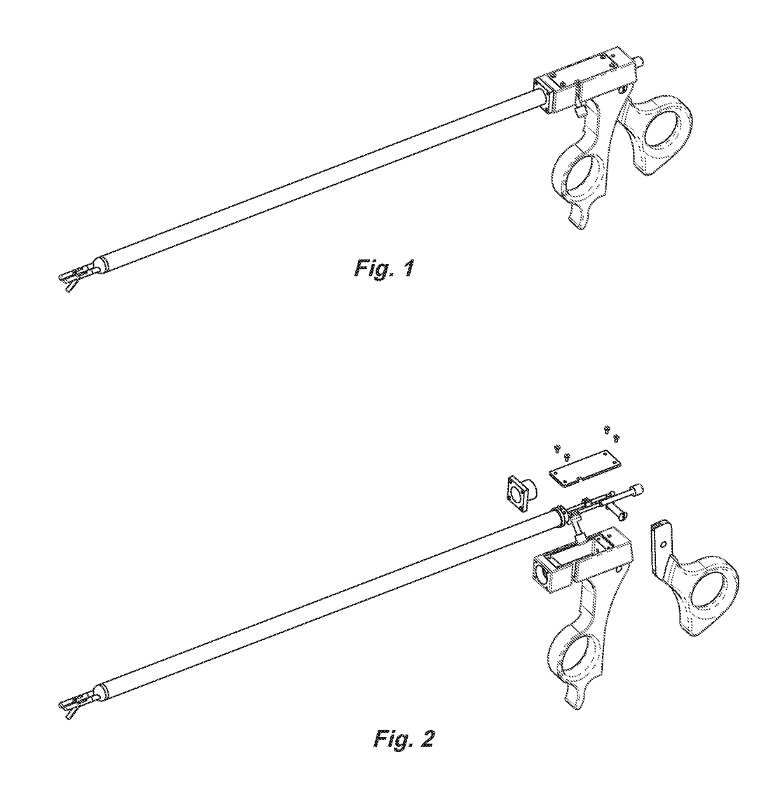

FIG. 1 is a perspective view of the entire instrument from the left side.

FIG. 2 is an exploded view of the handle end of the instrument.

Sheet 2

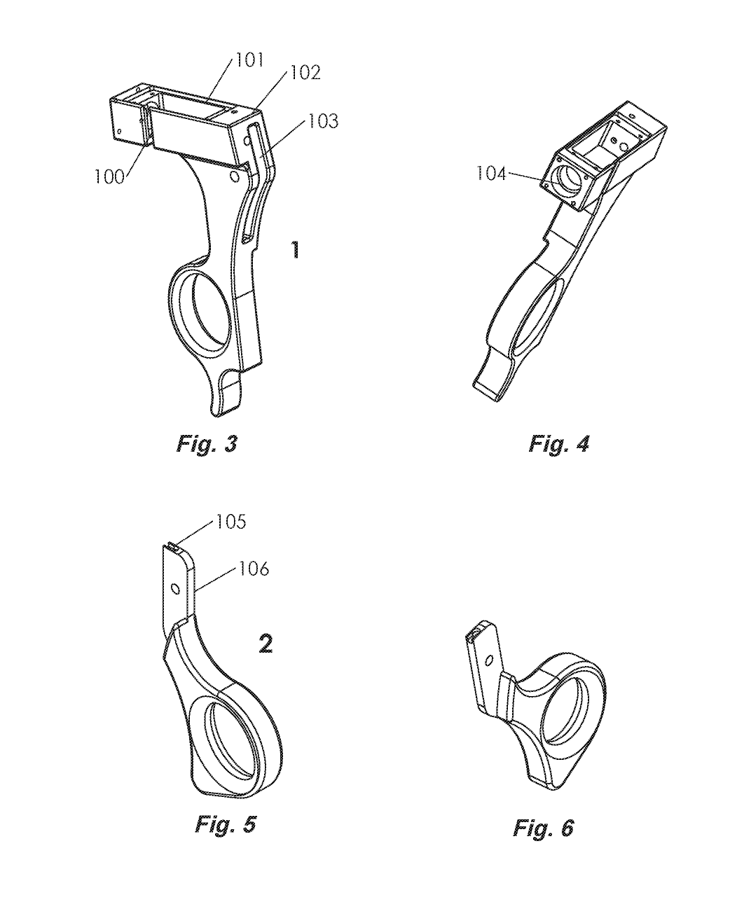

FIG. 3 is a perspective view of the stationary handle. The feature 100 is a vertical slot on the left side of the chamber 101, for the rotating lever. Feature 102 is sight hole on top of the handle to help installation of the underlying ball joint. 103 is vertical slot on the backside for hiding the upper limb of the movable handle.

FIG. 4 is another perspective view of the stationary handle. 104 is a large opening for entry of the rear end of the main sheath sub-assembly through the front of the chamber.

FIG. 5 is a perspective view of the movable handle. 105 is the receptacle for the ball to form the ball-joint. 106 is the narrow upper stem of the movable handle.

FIG. 6 is another perspective view of the movable handle.

Sheet 3

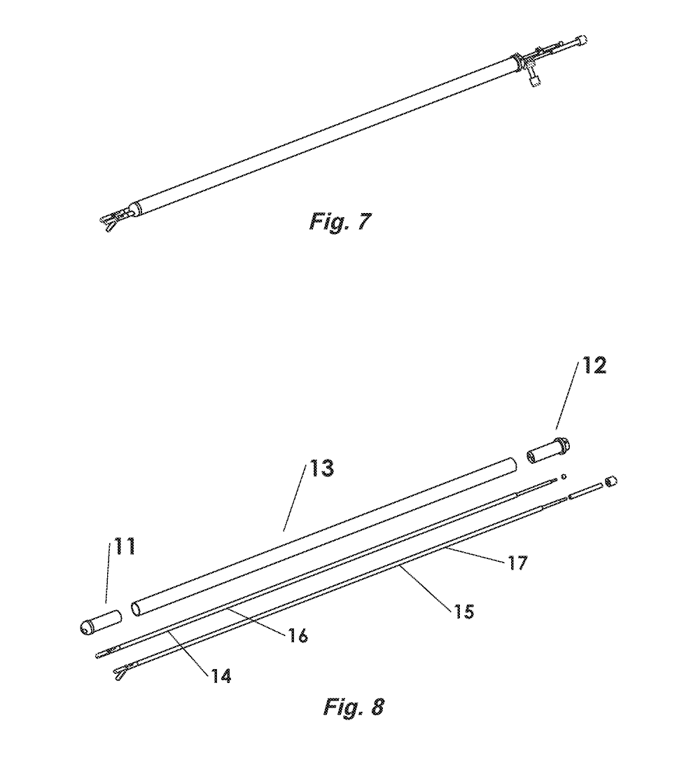

FIG. 7 is a perspective view of the main sheath sub-assembly,

FIG. 8 is an exploded view of the main sheath sub-assembly, showing its main components.

Sheet 4

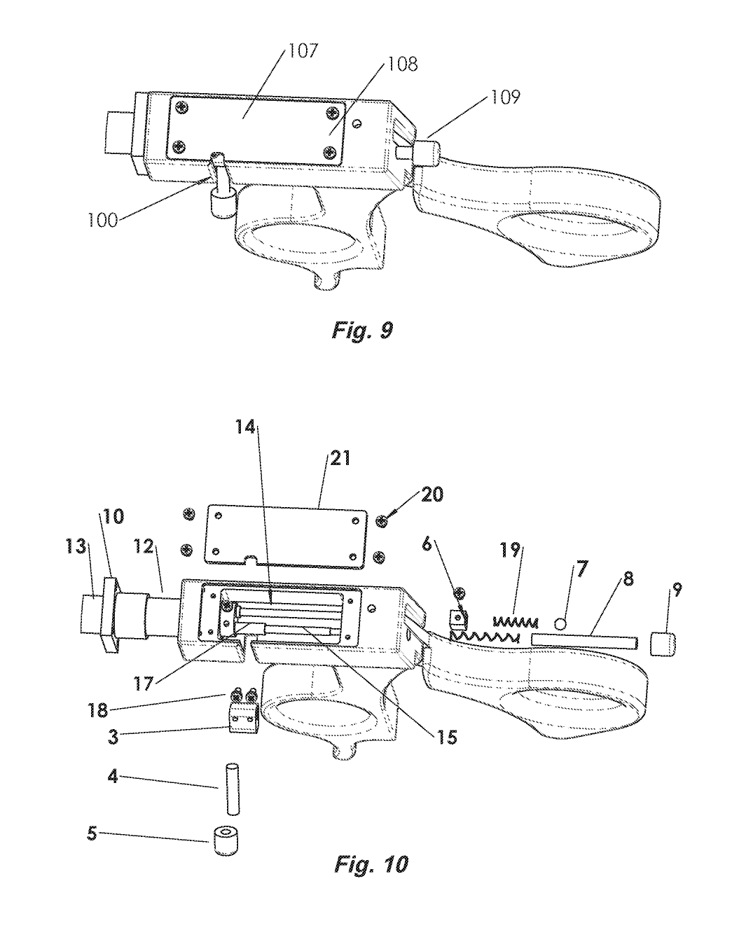

FIG. 9—shows the mechanisms inside the chamber, viewed from the top of the stationary handle.

FIG. 10—shows an exploded view of the numerous parts and mechanisms inside the chamber.

Sheet 5

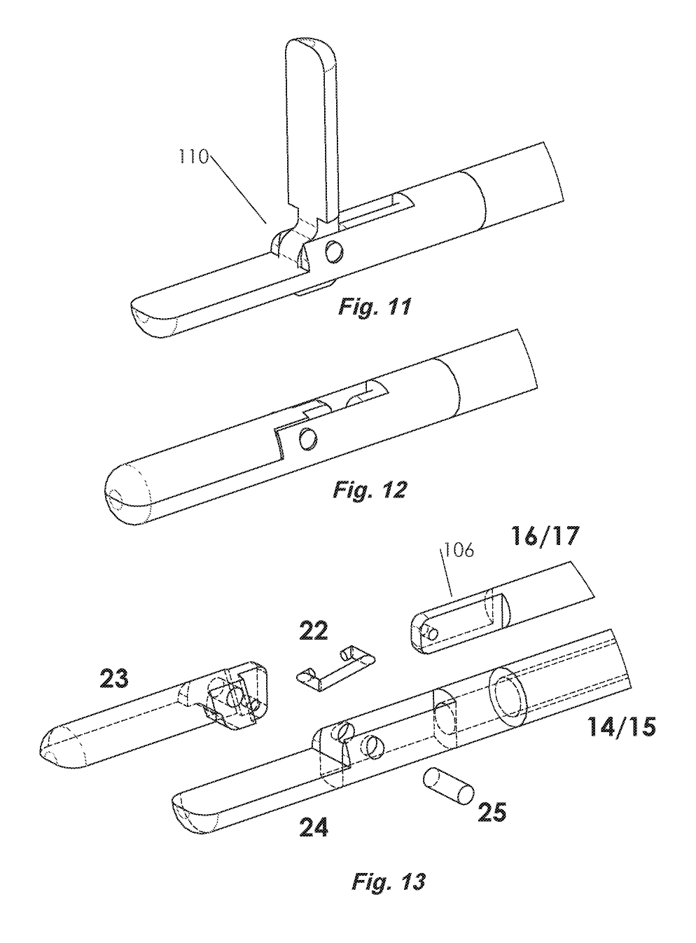

FIG. 11—shows the upper jaw opened to 90 degrees. Feature 110 indicates this unique capability.

FIG. 12—shows the upper jaw fully closed.

FIG. 13—shows the components of the Jaws Exploded. Please see the section on Part Names and Numbers. Note, the rear end of the fixed jaw is plugged into the lumen of the 3.0 mm. sheath. The jaws are identical on the 2 sides, but their rear ends are different.

Sheet—6

Demonstrates the steps in the tying process using this instruments:

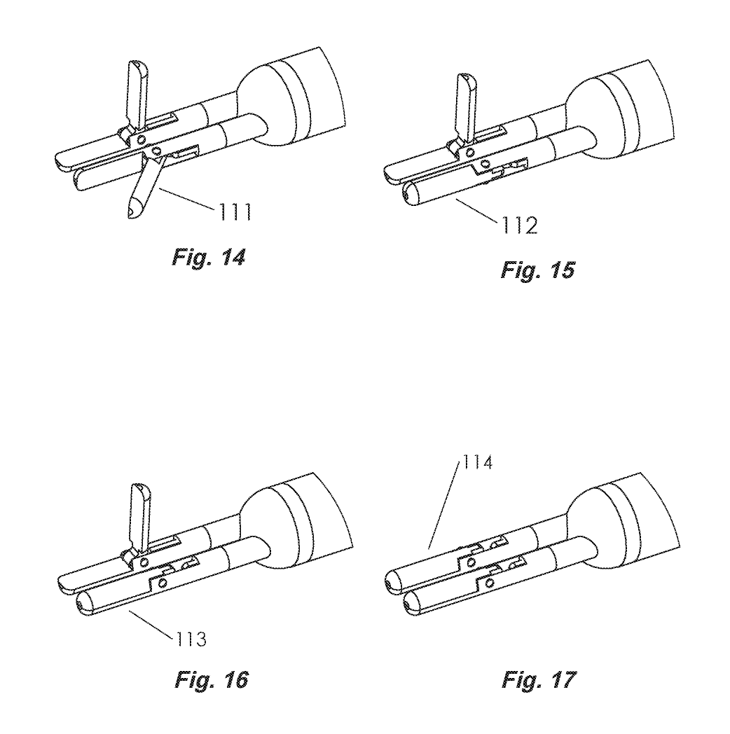

FIG. 14—shows the donor jaws positioned in the vertical plane and opened to 45 degrees, as indicated by 111, and the recipient jaws positioned in the horizontal plane and opened to 90 degrees.

FIG. 15—shows the donor jaws closed but remaining in the vertical plane, and the recipient jaw still open to 90 degrees, as indicated by 112.

FIG. 16—shows the donor jaws remaining closed and now rotated 90 degrees to the right, with the recipient jaw remaining open to 90 degrees, as indicated by 113.

FIG. 17—shows the upper jaw of the recipient grasper now closed, and taking over the head end of the suture, as indicated by 114.

Sheet 7

FIG. 18 shows a basic suture tying diagram, with naming of the different parts of a suture. 1=Head End. 2=Tail End. 3=Head Strand. 4=Tail Strand. The Head Strand is shown as thin line and the Tail Strand is shown as thick line. The Direction Arrows are self-explanatory.

FIG. 19 shows exactly how a knot or tie is made. The names of the different parts of a suture, and the direction arrows are also shown.

Sheet 8

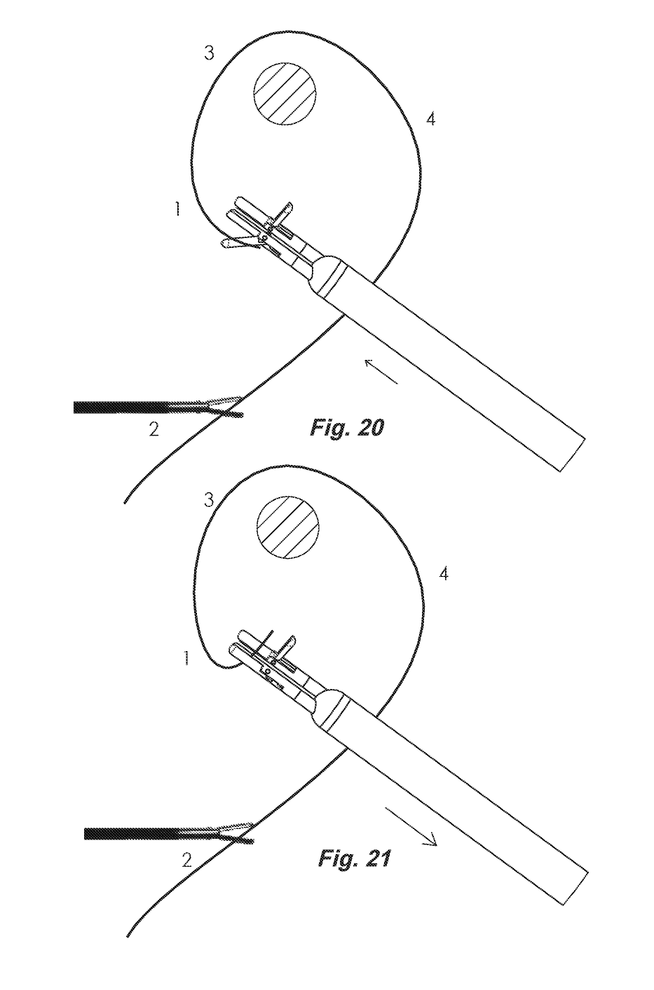

FIG. 20 shows the starting position when tying a knot, showing the tail end being first held straight with a second regular instrument in the surgeon's left hand, and showing the knot tying instrument first going forwards, crossing over the tail strand, grasping the head end.

FIG. 21 shows the knot tying instrument having grasped the head end of the suture with the donor grasper, ready to pull back over the tail strand.

Sheet 9

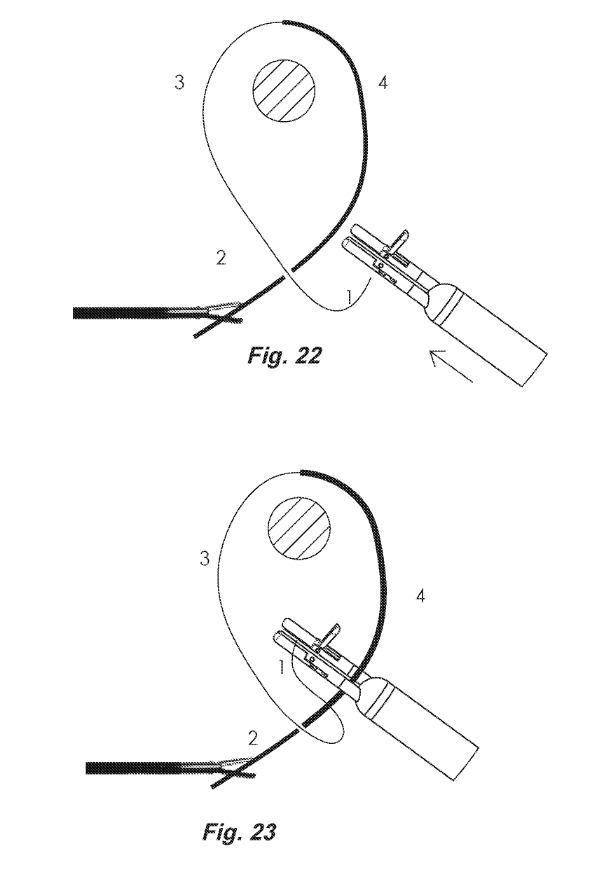

FIG. 22 shows the knot tying instrument now pushing forwards to trap the tail strand in the slot between the two grasper tips, with the donor grasper on the underside, below the tail strand, and the recipient grasper above the tail strand. This step must occur exactly as stated, namely the head end must first be grasped by the donor grasper before the entrapping of the tail strand, otherwise it would not work.

FIG. 23 shows a close-up view of the next step which is rotation of the jaws of the donor grasper 90 degrees to the right, which will then place the head end of the suture on top of the lower jaw of the recipient grasper. The initial orientation of the jaws of the donor grasper in the vertical plane, and its subsequent rotation, are absolutely necessary. If the jaws of the donor grasper were in the horizontal plane to start with, then the suture grasped in its jaws would block entry of the tail strand into the slot, which is the reason why the concept of two parallel graspers, with one of them sliding in and out—was not chosen.

Sheet 10

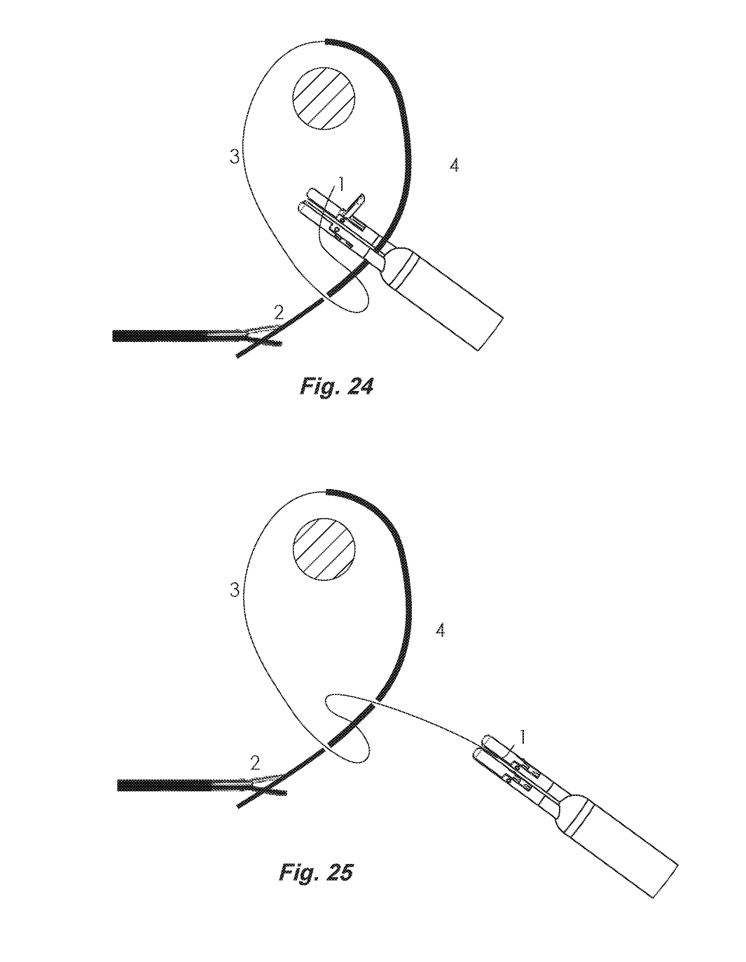

FIG. 24 shows the upper jaw of the recipient grasper closing down.

FIG. 25 shows the take away, and the formation of the knot, with direction arrows and numbers of consecutive steps.

Sheet 11

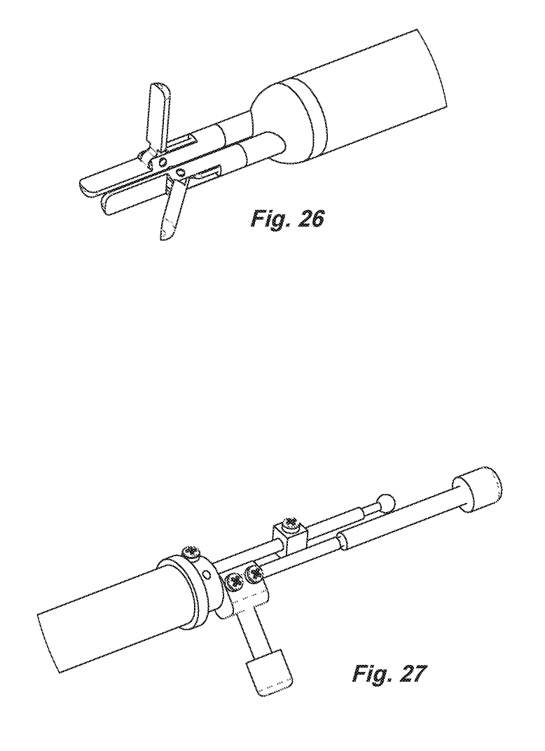

FIG. 26 shows a close-up view of the front end of the instrument, at the start of the tying-process.

FIG. 27 shows a close-up view of the rear end of the instrument, without the handles or the springs.

Sheet 12

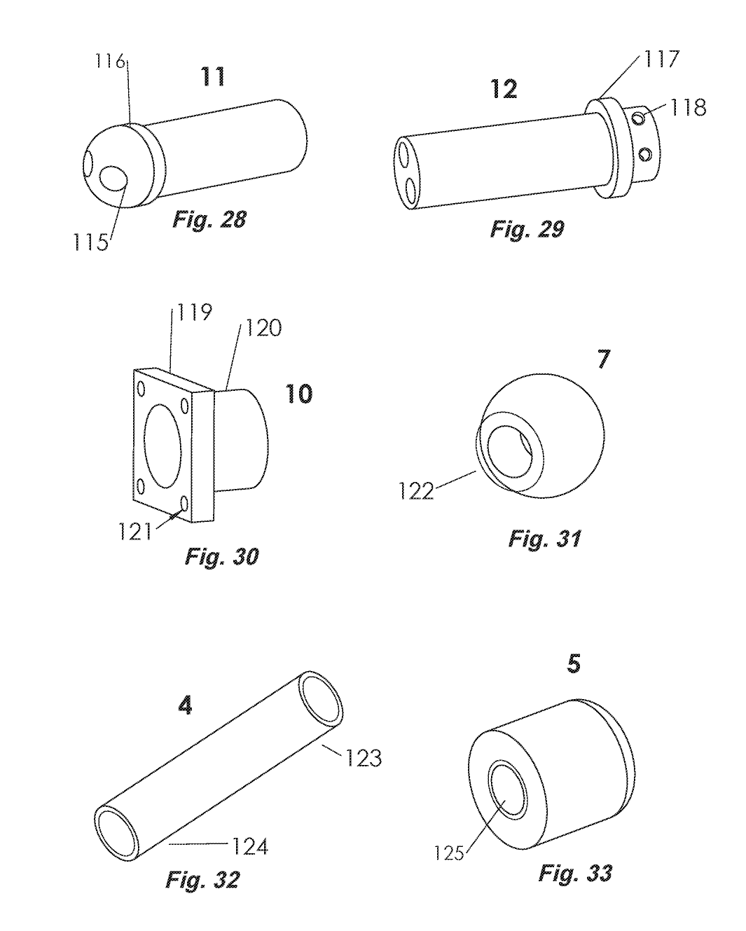

FIG. 28 shows the Front Spacer, 11.

FIG. 29 shows the Rear Spacer, 12

FIG. 30 shows the Retaining Flanged Nut, 10.

FIG. 31 shows the Ball of the ball joint, 7

FIG. 32 shows the Lever, used for rotation, 4

FIG. 33 shows the Button for the lever, 5

Sheet 13

FIG. 34 shows the Adaptor-2, for the Lever, #3. Feature 126 are holes for mini-screws which bind the Adaptor-2 to Sheath-2. 127 is for passage of Sheath-2. 128 is for attachment of the rotation Lever.

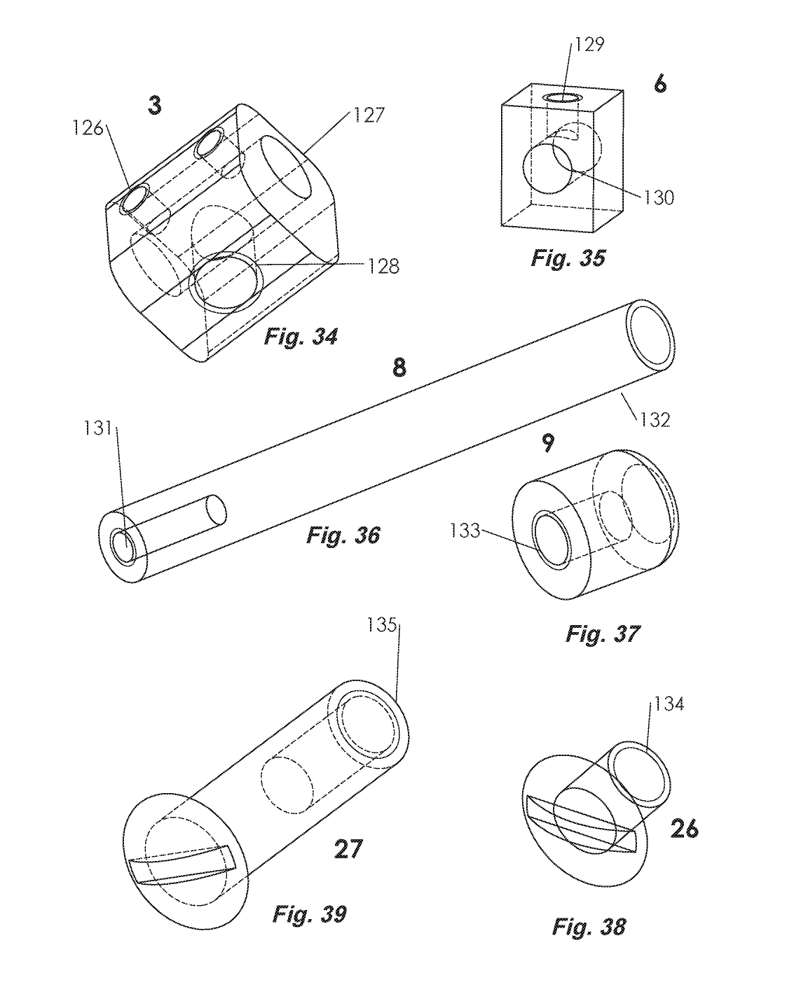

FIG. 35 shows the Adaptor-1, #6, acting as a Stop for the Spring on the Recipient Grasper. 129 is the mini-screw. 130 is for passage of the Rod-1.

FIG. 36 shows the Rod Extension #8 for the Donor Grasper. The end 131 mates with the male end of Rod-2. The end 132 mates with the end 133 of the Push Button #9.

FIG. 37 shows the Push Button #9 for the Rod Extension.

FIG. 38 shows the Male Hinge Bolt #26. The end 134 mates with 135 of the Female Hinge Bolt

FIG. 39 shows the Female Hinge Bolt #27, which is the longer one.

Sheet 14

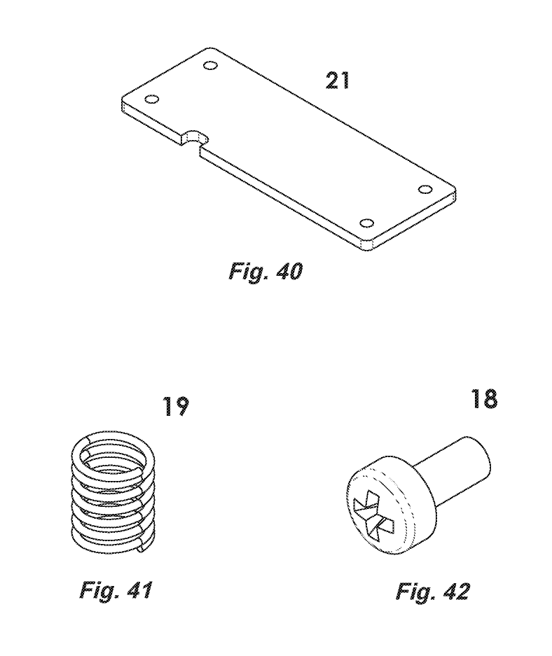

FIG. 40 shows the Lid #21. The notch is for the rotation lever.

FIG. 41 shows a sample of the Compression Spring used #19, which are 3 mm OD

FIG. 42 shows a sample of a Mini-Screw #18, which are M1.4, in 3 different lengths.

Sheet 15

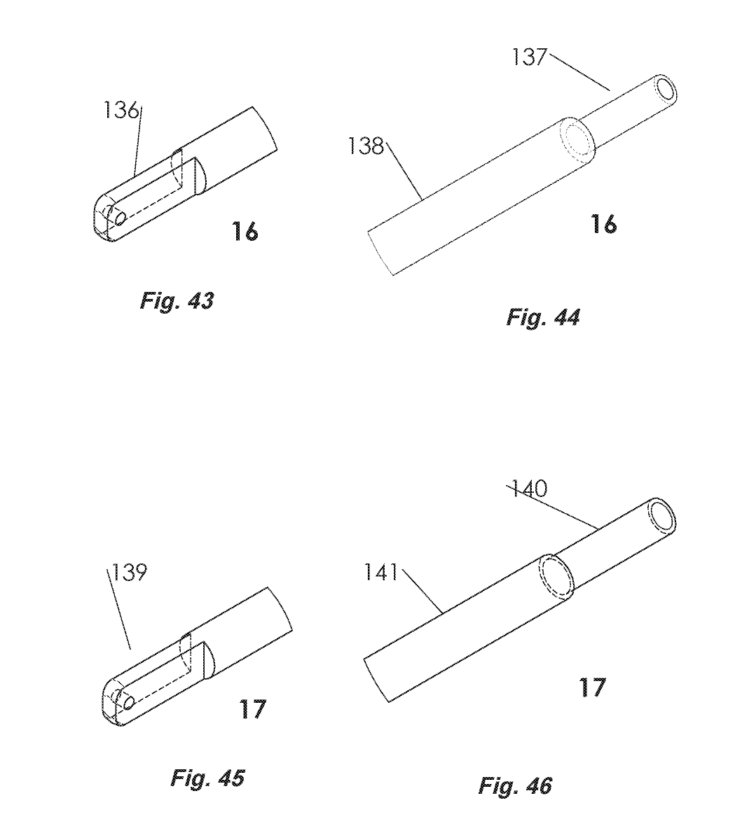

FIG. 43 shows the shape of the distal end of Rod-1, # 16. 136 shows the end is a narrow flat plate with a hole for the jaw pin.

FIG. 44 shows the proximal end of Rod-1, #16, which is M2.0. 137 shows the end is a small male thread, M1.3, which screws into the Ball.

FIG. 45 shows the distal end of Rod-2, #17, which is identical to that of Rod-1. 139 shows the end is a narrow flat plate with a hole for the jaw pin.

FIG. 46 shows the proximal end of Rod-2, 140 which ends in a male thread with a size of M1.6 to be mated with female thread on the proximal end of the Rod-Extension with a size of M3.0.

### DETAILED DESCRIPTION OF THE PREFERRED EMBODIMENT

The assembly begins with assembling the two jaws, followed by combining them with the rods and the sheaths to form the two “grasper-sub-assemblies”. The Front-Spacer is then mounted on to them, followed by the main sheath from the rear to the front. The Rear-Spacer is then mounted from the rear, completing the “Main Sub-assembly”. Adhesives are applied. Note, the main sheath is trapped between the stops on the two spacers. Sheath-1 is then locked down with the mini-screw onto the rear spacer. Adaptor-1, Spring-1 and the ball are mounted onto Rod-1. Adaptor-2, and Spring-2 are mounted onto Rod-2. The Adaptors are locked down. The completed “Main Sub-assembly” is then gently inserted into the front end of the Chamber located on the top of the Stationary Handle. The Ball will go through the rear wall of the Chamber. The Rod-Extension with its Button is inserted from the rear of the chamber, and attached to the end of Rod-2. The Retaining Flange Nut is then firmly attached. The Main Shaft is attached to the Retaining Flange Nut with adhesives. The Lever with its Button is screwed onto Adaptor-2. The mini-screws on Adaptor-2 are adjusted to allow the Jaw-2 to be orientated to the vertical position, with the Lever in the maximal down position.

The following features are essential to this device:

(1) It is necessary to have two mini-graspers side by side, within the same instrument, in order to pass the head end of the suture from one to the other, and to trap the tail end of the suture in the gap between them.

(2) One of the graspers must have a regular scissors type of handle, in order to provide the necessary power to the jaw-grip in the final take-away.

(3) The second grasper must be able to rotate. Therefore, it cannot have a handle, and its gripping power must come a compression spring.

(4) A finger operated 360-degree of rotation of the shaft and jaws is an established prior art. However, this is not possible when combined with a second instrument. The present design is unique in permitting only 90-degree rotation, which is exactly what is required.

(5) The present design utilizes the thumb for activation of the lever, but this can be easily changed to utilize the index finger.

(6) In Rod-1, a compression spring is used to keep the upper jaw open to 90 degrees, make it ready to receive the passing head end of the suture.

(7) In Rod-2, a compression spring is used to keep the jaws closed, to hold onto the head end of the suture during rotation. The jaws are opened by forward pressure from the thumb on the Push Button.

(8) To tie a knot with this instrument, it is necessary to also use a second regular instrument in the surgeon's other hand.

(9) The top of the Movable Handle bearing the receptacle for the Ball Joint, is intentionally hidden within the body of the Stationary Handle, to avoid it interfering with the thumb, when the thumb pushes forward the Push Button on Rod-2.

A compression spring is used on Rod-1 to keep its upper jaw open to 90 degrees, which is necessary in order to receive the passing head end of the suture. This is a light spring simply to keep its upper jaw open to 90 degrees all the time. It is easily closed when the movable handle is lightly squeezed during entry into and withdrawal from the abdominal cavity.

A heavier compression spring is used on Rod-2, to keep its jaws closed at all times. When grabbing the head end of the suture, these jaws require to be first opened by pushing forwards with the thumb.

The rotation and counter-rotation of the donor grasper are performed by flicking the thumb on the lever.

1. An instrument for tying a knot in a suture laparoscopically, involving passing the head end of a suture from a donor grasper to an adjacent recipient grasper behind and around the tail end of the same said suture, comprising:
the shafts of two small diameter laparoscopic graspers housed within a common external sheath, with one being a regular grasper having a customary scissors type of handle, and acting as a recipient grasper; and a second grasper without a handle but rotatable, and acting as a donor grasper, and further comprising:
having a narrow gap between the tips of the said two graspers for trapping the tail strand of the said suture; the said donor grasper having a non-movable inner/lower jaw enabling its rotation; the said recipient grasper having both a straight lower jaw and a single acting 90 degree movable upper jaw providing a platform for reception of the passing suture; having the jaws of the said donor grasper in the vertical plane in the resting state and able to rotate through 90 degrees after grasping the head end of the suture in it's jaws; having a compression spring holding closed the upper jaw of the said donor grasper; and
having a compression spring holding open to 90 degrees the upper jaw of the said recipient grasper.

2. A method of tying a knot in a free length of suture laparoscopically with the instrument of claim 1, wherein the head end of a suture is passed behind and around its tail strand from a donor grasper to an adjacent recipient grasper thus encircling the said tail strand forming a knot, and comprising the steps of:
(1) grasping the head end of a suture with the jaws of the said donor grasper and pulling it back over the said tail strand;
(2) advancing the said instrument forwards to trap the said tail strand in the slot between the tips of the two said graspers, with the said donor grasper under the said tail strand;
(3) rotating the said donor grasper and its jaws 90 degrees clockwise, thus placing the head end of the said suture on top of the straight lower jaw of the said recipient grasper;
(4) closing down the upper jaw of the said recipient grasper, taking over the said head end of the said suture, and pulling away forming the knot.
